SEQUENCE LISTING (3 sequences)

SEQ ID NO: 1 - DNA, 801 bp, Homo sapiens
gagatggcagagttgatcttttatcatctcttggtgaaagcccagtaacataagactgctctaggctgtctgcatgcctgtctatctaaattaactagcttggttgctgaacaccaggttaggctctcaaattaccctctgattctgatgtggcctgagtgtgacagttaattattgggaatatcaaaacaattacccagcatgatcatgtattatttaaacagtcctgacagaactgtacctttgtgaacagtgcttttgattgttctacatggcatattcacatccattttcttccacagggtgatcttctgttctagggagaaagtgtctcatttgcagaaaggaaaggcacctgcggtatttgtgaaatgccatgacaagtctctgaataagaagtsaggctggtgagcattctgggctaaagctgactgggcatcctgagcttgcaccctaagggaggcagcttcatgcattcctcttcaccccatcaccagcagcttgccctgactcatgtgatcaaagcattcaatcagtctttcttagtccttctgcatatgtatcaaatgggtctgttgctttatgcaatacttcctctttttttctttctcctcttgtttctcccagcccggaccttcaacccaggcacacattttaggttttattttactccttgaactacccctgaatcttcacttctccttttttctctactgcgtctctgctgactttgcagatgccatctgcagagcatgtaacacaagtttagtagttgccgttctggctgtgggtgcagctctt

SEQ ID NO: 2 - DNA, 625 bp, Homo sapiens
gcgaggagcggacagggaaagagacctcaacatgaccagggatgggaggagagaactccattacttagaaacagtggctgctatgctgggaagtgtggactgttacagtgggctctaattccaggaggaacaaaaagacccsgcatatcaccatgagtcagtcctctgagtcgccatgacccatgaccttgctaaacaagtgtggggctgtcacagccttaagcagcacatctcaagtctcacattctccagtgctaagaaaacagctacacagacagaatgctaaggagagctcagaagtagaaaacccttaagagtccccccttccttagataggaaaactgaggcccagagaggcaaacaatgggtcagtatgtgtgtctaaacttatctaaacatagaagaggtatagtaaaaatacggtattagaatctgatgggagcaccatcgtatacgcagtctgtagttgaccagaacattggtatgcggcacatgactgtagatcttctcattaataataggcaacctggtcaggtgcacgaagtctagggttcagaatccaacaggctcaaattcaagtccagctcagccacgtggctgagtgctgtctgaacctcagcgtcct

SEQ ID NO: 3 - DNA, 501 bp, Homo sapiens
ttctgagtcatttattttccttagctttttccactcaaattaagggcaagcgaaaaagtaataatttggcattctttaagcctacagaatgtgattctttcacttgtttattacactggctcgtggacagaacaatttgaaaagtgaaagaattattttggtaaaagattttgctttacttttcgaagcattatttttttaaagagtgttttactccaacgattgaaacattttcctatttaaatttcatygttagaatcacaggaggcaaaaaatggaacggttgaatgaaattttactctttctgtgaaagaaaatccacagagttgttgcctccgttgtagttggtgggccccgttagcattggatgcctttgccaaatggttcatgtggacacacaaaggcaaacagatctgccatcgatcgcagatttctgtagaaacacggatgtgcatgtgcagattcccttttgcaggtattaaaaataattaaaaatagtcc

(19) United States
(12) Patent Application Publication
(10) Pub. No.: US 2012/0088245 A1
(43) Pub. Date: Apr. 12, 2012

(54) METHODS OF DIAGNOSING INSULIN RESISTANCE AND SENSITIVITY

(75) Inventors: Jerome I. Rotter, Los Angeles, CA (US); Kent D. Taylor, Ventura, CA (US); Xiuqing Guo, Santa Monica, CA (US); Mark O. Goodarzi, Los Angeles, CA (US); Yii-Der I. Chen, Saratoga, CA (US); Leslie Raffel, Los Angeles, CA (US); Thomas A. Buchanan, Los Angeles, CA (US); Anny Xiang, Los Angeles, CA (US)

(73) Assignee: CEDARS-SINAI MEDICAL CENTER, Los Angeles, CA (US)

(21) Appl. No.: 13/126,695

(22) PCT Filed: Oct. 30, 2009
(86) PCT No.: PCT/US09/62835

§ 371 Date: Dec. 23, 2011

Related U.S. Application Data
(60) Provisional application No. 61/110,473, filed on Oct. 31, 2008.

(51) Int. Cl. C12Q 1/68

(52) U.S. Cl. 435/6.12

(57) ABSTRACT

Methods of diagnosing susceptibility to metabolic insulin resistance and other related conditions are disclosed. The method provides means of diagnosing susceptibility to insulin resistance in Hispanic Americans by determining the presence of a risk haplotype at the LPL locus, the LPIN1 locus, and/or elevated levels of gamma-glutamyl transferase.

### GOVERNMENT RIGHTS

This invention was made with U.S. Government support by NIH grants HL069794 and HL088457. The U.S. Government may have certain rights in this invention.

### FIELD OF THE INVENTION

The invention relates generally to the fields of metabolism and metabolic traits and, more specifically, to genetic methods of diagnosing insulin resistance and sensitivity.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Metabolic syndrome affects an estimated 50 million people in the United States alone. Those with metabolic syndrome, also called insulin resistance syndrome, have an increased risk of diabetes, and diseases that are related to plaque build ups in artery walls such as coronary heart disease. Although the specific causes of metabolic syndrome are not completely understood, primary risk factors include abdominal obesity, and insulin resistance where the body is unable to use insulin efficiently. Elevated liver enzyme levels such as gamma glutamyl transferase (GGT), likely a reflection of fatty liver, have also been associated with insulin resistance and metabolic syndrome. Family studies have shown that both the metabolic syndrome and liver enzymes are heritable, with heritability and co-heritability analyses indicating significant evidence for a genetic contribution to liver enzyme levels.

Although there have been some associations found between risk factors and metabolic traits, the exact cause and contribution factors for many of the metabolic diseases are largely unknown. Thus, there is need in the art to determine genes, allelic variants, biological pathways, and other factors that contribute to metabolic traits, including but not limited to metabolic syndrome and insulin resistance.

### SUMMARY OF THE INVENTION

Various embodiments include a method of diagnosing susceptibility to insulin resistance in an individual, comprising determining the presence or absence in the individual of a risk variant at the Lipoprotein Lipase (LPL) genetic locus and/or Lipin-1 (LPIN1) genetic locus, determining the presence or absence in the individual of an elevated level of a marker for fatty liver, and diagnosing susceptibility to insulin resistance in the individual based upon the presence of the risk variant at the LPL genetic locus and/or LPIN1 genetic locus and the presence of elevated level of the marker for fatty liver. In another embodiment, the risk variant at the LPL genetic locus comprises SEQ. ID. NO.: 1. In another embodiment, the risk variant at the LPIN1 genetic locus comprises SEQ. ID. NO.: 2 and/or SEQ. ID. NO.: 3. In another embodiment, the individual is Hispanic American. In another embodiment, the marker for fatty liver comprises GGT.

Other embodiments include a method of determining a low probability of developing insulin resistance in an individual, comprising determining the presence or absence in the individual of a protective haplotype at the Lipin-1 (LPIN1) genetic locus, determining the presence or absence of a low level of expression of an inflammatory marker, and diagnosing a low probability of developing insulin resistance in the individual based upon the presence of a protective haplotype at the LPIN1 genetic locus and the presence of a low level of expression of the inflammatory marker. In another embodiment, the inflammatory marker comprises TNFR1. In another embodiment, the protective haplotype at the LPIN1 genetic locus comprises SEQ. ID. NO.: 2 and/or SEQ. ID. NO.: 3. In another embodiment, the individual is Hispanic American.

Other features and advantages of the invention will become apparent from the following detailed description, taken in conjunction with the accompanying drawings, which illustrate, by way of example, various embodiments of the invention.

### DESCRIPTION OF INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al. Dictionary of Microbiology and Molecular Biology 2nd ed., J. Wiley & Sons (New York, N.Y. 1994); March, Advanced Organic Chemistry Reactions, Mechanisms and Structure 4th ed., J. Wiley & Sons (New York, N.Y. 1992); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described.

As used herein, “GGT” means gamma-glutamyl transferase.

As used herein, “LE” means liver enzyme.

As used herein, “HA” means Hispanic American. As used herein, Hispanic American means all American persons of Mexican, Puerto Rican, Cuban, Central, Latin or South American, Portuguese, or other Spanish culture of origin.

As used herein, “LPL” means lipoprotein lipase. An example of LPL SNP rs328 is described herein as SEQ. ID. NO.: 1. As readily apparent to one of skill in the art, other examples of sequences may also be used to also capture the same allele, such as the complementary strand sequence.

As used herein, “IR” means insulin resistance.

As used herein, “LPIN1” means lipin-1. An example of LPIN1 SNPs rs893347 and rs11524 are described herein as SEQ. ID. NO: 2 and SEQ. ID. NO.: 3, respectively. As apparent to one of skill the in the art, additional examples of sequences may also be used to capture the same allele.

As used herein, “TNFR1” and “TNFR2” means tumor necrosis factor receptor 1 and 2, respectively.

As used herein, the term “biological sample” means any biological material from which nucleic acid molecules can be prepared. As non-limiting examples, the term material encompasses whole blood, plasma, saliva, cheek swab, or other bodily fluid or tissue that contains nucleic acid.

As disclosed herein, the inventors studied the role of genetic variants in the LPL gene on GGT levels using 618 non-diabetic offspring from 160 HA families ascertained through a proband with hypertension. GGT was measured by enzymatic colorimetry. Six single nucleotide polymorphisms (SNPs) known to be in the same block in the LPL gene were genotyped in these samples. The generalized transmission disequilibrium test as implemented in the QTDT program was used in the association analysis. To avoid false positives derived from population stratification, the within family variance component was used for the association testing. After adjusting for age, sex, and body mass index, significant association with GGT was found for SNP Ser447Stop/rs328 (p=0.019). Haplotype analysis revealed that the SNP was located at the fourth most common haplotype (GAGGGG), which was also significantly associated with decreased GGT (28.5±2.6 vs 32.2±1.2 U/L, p=0.009). This haplotype has been previously reported as significantly associated with IRS in HA families recruited through CAD probands (Goodarzi et al., Diabetes, 53:214-220, 2004). These results show that the LPL gene is a common genetic determinant for LEs and IRS in the Hispanic American population.

In one embodiment, the present invention provides a method of diagnosing susceptibility in an individual to insulin resistance by determining the presence or absence of a risk haplotype at the LPL locus and elevated levels of gamma-glutamyl transferase, where the presence of a risk haplotype at the LPL locus and elevated levels of gamma-glutamyl transferase are indicative of susceptibility to insulin resistance. In another embodiment, the risk haplotype includes SNP Ser447Stop/rs328. In another embodiment, the individual is Hispanic American.

In another embodiment, the present invention provides a method of treating insulin resistance in an individual by determining the presence of a risk haplotype at the LPL locus and elevated levels of gamma-glutamyl transferase, and treating the insulin resistance.

In another embodiment, the present invention provides a method of diagnosing a decreased likelihood of susceptibility to insulin resistance relative to a healthy individual by determining the presence or absence of a protective haplotype at the LPL locus and decreased levels of gamma-glutamyl transferase in a subject, where the presence of a protective haplotype at the LPL locus and decreased levels of gamma-glutamyl transferase is indicative of a decreased likelihood of susceptibility to insulin resistance relative to a healthy individual. In another embodiment, the protective haplotype at the LPL locus includes the fourth most haplotype of GAGGGG. In another embodiment, the subject is Hispanic American.

As further disclosed herein, the inventors performed a study to determine whether variants in the gene for lipin-1 (LPIN1) were associated with the liver enzyme gamma glutamyl transferase (GGT, a marker for fatty liver) or inflammatory markers (C-reactive protein, serum tumor necrosis factor (TNF) receptor 1 (TNFR1) and receptor 2 (TNFR2)). The study cohort consisted of 618 non-diabetic offspring from 160 Hispanic-American families ascertained through a proband with hypertension. Two SNPs on opposite ends of the LPIN1 gene were genotyped, haplotypes constructed, and tested for association using generalized estimating equations (GEE1) to account for familial correlation, adjusting for age, sex, and BMI. Haplotype 1 (most common haplotype) was associated with an increase in GGT (haplotype carriers 31.7±1.1 vs non-carriers 29.8±5.1 U/L, p. 0.026). SNP rs11524 was associated with decreased TNFR1 (1.77±0.035 vs 1.83±0.020 ng/mL, dominant model, p=0.029). Haplotype 2, which carries rs11524, exhibited the same association. Computational modeling suggests that rs11524 alters an exonic splicing silencer sequence (Ong K L, et al. Am J Hypertens 2008; 21:539-45). Consistent with predictions based on the biology of lipin-1, variants in the LPIN1 gene modulates liver function and inflammation.

In one embodiment, the present invention provides a method of diagnosing insulin resistance by determining the presence or absence of a risk haplotype at the LPIN1 locus and an elevated level of GGT, where the presence of the risk haplotype at the LPIN1 locus and/or elevated level of GGT is indicative of insulin resistance. In another embodiment, the risk haplotype is LPIN1 haplotype 1. In another embodiment, the individual is Hispanic American.

In another embodiment, the present invention provides a method of diagnosing a decrease in likelihood of susceptibility to insulin resistance relative to a healthy individual by determining the presence or absence in a subject of a protective haplotype at the LPIN1 locus and a low level of expression of TNFR1, where the presence of a protective haplotype at the LPIN1 locus and/or a low level of expression of TNFR1 is indicative of a decrease in likelihood of susceptibility to insulin resistance relative to a healthy individual. In another embodiment, the protective haplotype is LPIN1 haplotype 2. In another embodiment, the protective haplotype includes SNP rs11524. In another embodiment, the subject is Hispanic American.

There are many techniques readily available in the field for detecting the presence or absence of enzymes, proteins, polypeptides or other biomarkers, including protein microarrays. For example, some of the detection paradigms that can be employed to this end include optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

Similarly, there are any number of techniques that may be employed to isolate and/or fractionate enzymes and/or biomarkers. For example, a biomarker may be captured using biospecific capture reagents, such as antibodies, aptamers or antibodies that recognize the biomarker and modified forms of it. This method could also result in the capture of protein interactors that are bound to the proteins or that are otherwise recognized by antibodies and that, themselves, can be biomarkers. The biospecific capture reagents may also be bound to a solid phase. Then, the captured proteins can be detected by SELDI mass spectrometry or by eluting the proteins from the capture reagent and detecting the eluted proteins by traditional MALDI or by SELDI. One example of SELDI is called “affinity capture mass spectrometry,” or “Surface-Enhanced Affinity Capture” or “SEAC,” which involves the use of probes that have a material on the probe surface that captures analytes through a non-covalent affinity interaction (adsorption) between the material and the analyte. Some examples of mass spectrometers are time-of-flight, magnetic sector, quadrupole filter, ion trap, ion cyclotron resonance, electrostatic sector analyzer and hybrids of these.

Alternatively, for example, the presence of biomarkers such as enzymes and polypeptides maybe detected using traditional immunoassay techniques. Immunoassay requires biospecific capture reagents, such as antibodies, to capture the analytes. The assay may also be designed to specifically distinguish protein and modified forms of protein, which can be done by employing a sandwich assay in which one antibody captures more than one form and second, distinctly labeled antibodies, specifically bind, and provide distinct detection of, the various forms. Antibodies can be produced by immunizing animals with the biomolecules. Traditional immunoassays may also include sandwich immunoassays including ELISA or fluorescence-based immunoassays, as well as other enzyme immunoassays.

Prior to detection, biomarkers such as enzymes may also be fractionated to isolate them from other components in a solution or of blood that may interfere with detection. Fractionation may include platelet isolation from other blood components, sub-cellular fractionation of platelet components and/or fractionation of the desired biomarkers from other biomolecules found in platelets using techniques such as chromatography, affinity purification, 1D and 2D mapping, and other methodologies for purification known to those of skill in the art. In one embodiment, a sample is analyzed by means of a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there.

The methods may include the steps of obtaining a biological sample containing nucleic acid from the individual and determining the presence or absence of a SNP and/or a haplotype in the biological sample. The methods may further include correlating the presence or absence of the SNP and/or the haplotype to a genetic risk, a susceptibility for metabolic syndrome and metabolic traits thereof including but not limited to insulin resistance, as described herein. The methods may also further include recording whether a genetic risk, susceptibility for metabolic syndrome and metabolic traits thereof including but not limited to insulin resistance exists in the individual. The methods may also further include a prognosis of metabolic syndrome and metabolic traits thereof based upon the presence or absence of the SNP and/or haplotype. The methods may also further include a treatment of metabolic syndrome and metabolic traits thereof based upon the presence or absence of the SNP and/or haplotype.

In one embodiment, a method of the invention is practiced with whole blood, which can be obtained readily by non-invasive means and used to prepare genomic DNA, for example, for enzymatic amplification or automated sequencing. In another embodiment, a method of the invention is practiced with tissue obtained from an individual such as tissue obtained during surgery or biopsy procedures.

A variety of methods can be used to determine the presence or absence of a genetic variant allele or haplotype. As an example, enzymatic amplification of nucleic acid from an individual may be used to obtain nucleic acid for subsequent analysis. The presence or absence of a variant allele or haplotype may also be determined directly from the individual's nucleic acid without enzymatic amplification.

Analysis of the nucleic acid from an individual, whether amplified or not, may be performed using any of various techniques. Useful techniques include, without limitation, polymerase chain reaction based analysis, sequence analysis and electrophoretic analysis. As used herein, the term “nucleic acid” means a polynucleotide such as a single or double-stranded DNA or RNA molecule including, for example, genomic DNA, cDNA and mRNA. The term nucleic acid encompasses nucleic acid molecules of both natural and synthetic origin as well as molecules of linear, circular or branched configuration representing either the sense or antisense strand, or both, of a native nucleic acid molecule.

The presence or absence of a variant allele or haplotype may involve amplification of an individual's nucleic acid by the polymerase chain reaction. Use of the polymerase chain reaction for the amplification of nucleic acids is well known in the art (see, for example, Mullis et al. (Eds.), The Polymerase Chain Reaction, Birkhauser, Boston, (1994)).

A TaqmanB allelic discrimination assay available from Applied Biosystems may be useful for determining the presence or absence of a variant allele. In a TaqmanB allelic discrimination assay, a specific, fluorescent, dye-labeled probe for each allele is constructed. The probes contain different fluorescent reporter dyes such as FAM and VICTM to differentiate the amplification of each allele. In addition, each probe has a quencher dye at one end which quenches fluorescence by fluorescence resonant energy transfer (FRET). During PCR, each probe anneals specifically to complementary sequences in the nucleic acid from the individual. The 5′ nuclease activity of Taq polymerase is used to cleave only probe that hybridize to the allele. Cleavage separates the reporter dye from the quencher dye, resulting in increased fluorescence by the reporter dye. Thus, the fluorescence signal generated by PCR amplification indicates which alleles are present in the sample. Mismatches between a probe and allele reduce the efficiency of both probe hybridization and cleavage by Taq polymerase, resulting in little to no fluorescent signal. Improved specificity in allelic discrimination assays can be achieved by conjugating a DNA minor grove binder (MGB) group to a DNA probe as described, for example, in Kutyavin et al., “3′-minor groove binder-DNA probes increase sequence specificity at PCR extension temperature,” Nucleic Acids Research 28:655-661 (2000)). Minor grove binders include, but are not limited to, compounds such as dihydrocyclopyrroloindole tripeptide (DPI).

Sequence analysis also may also be useful for determining the presence or absence of a variant allele or haplotype.

Restriction fragment length polymorphism (RFLP) analysis may also be useful for determining the presence or absence of a particular allele (Jarcho et al. in Dracopoli et al., Current Protocols in Human Genetics pages 2.7.1-2.7.5, John Wiley & Sons, New York; Innis et al., (Ed.), PCR Protocols, San Diego: Academic Press, Inc. (1990)). As used herein, restriction fragment length polymorphism analysis is any method for distinguishing genetic polymorphisms using a restriction enzyme, which is an endonuclease that catalyzes the degradation of nucleic acid and recognizes a specific base sequence, generally a palindrome or inverted repeat. One skilled in the art understands that the use of RFLP analysis depends upon an enzyme that can differentiate two alleles at a polymorphic site.

Allele-specific oligonucleotide hybridization may also be used to detect a disease-predisposing allele. Allele-specific oligonucleotide hybridization is based on the use of a labeled oligonucleotide probe having a sequence perfectly complementary, for example, to the sequence encompassing a disease-predisposing allele. Under appropriate conditions, the allele-specific probe hybridizes to a nucleic acid containing the disease-predisposing allele but does not hybridize to the one or more other alleles, which have one or more nucleotide mismatches as compared to the probe. If desired, a second allele-specific oligonucleotide probe that matches an alternate allele also can be used. Similarly, the technique of allele-specific oligonucleotide amplification can be used to selectively amplify, for example, a disease-predisposing allele by using an allele-specific oligonucleotide primer that is perfectly complementary to the nucleotide sequence of the disease-predisposing allele but which has one or more mismatches as compared to other alleles (Mullis et al., supra, (1994)). One skilled in the art understands that the one or more nucleotide mismatches that distinguish between the disease-predisposing allele and one or more other alleles are preferably located in the center of an allele-specific oligonucleotide primer to be used in allele-specific oligonucleotide hybridization. In contrast, an allele-specific oligonucleotide primer to be used in PCR amplification preferably contains the one or more nucleotide mismatches that distinguish between the disease-associated and other alleles at the 3′ end of the primer.

A heteroduplex mobility assay (HMA) is another well known assay that may be used to detect a SNP or a haplotype. HMA is useful for detecting the presence of a polymorphic sequence since a DNA duplex carrying a mismatch has reduced mobility in a polyacrylamide gel compared to the mobility of a perfectly base-paired duplex (Delwart et al., Science 262:1257-1261 (1993); White et al., Genomics 12:301-306 (1992)).

The technique of single strand conformational, polymorphism (SSCP) also may be used to detect the presence or absence of a SNP and/or a haplotype (see Hayashi, K., Methods Applic. 1:34-38 (1991)). This technique can be used to detect mutations based on differences in the secondary structure of single-strand DNA that produce an altered electrophoretic mobility upon non-denaturing gel electrophoresis. Polymorphic fragments are detected by comparison of the electrophoretic pattern of the test fragment to corresponding standard fragments containing known alleles.

Denaturing gradient gel electrophoresis (DGGE) also may be used to detect a SNP and/or a haplotype. In DGGE, double-stranded DNA is electrophoresed in a gel containing an increasing concentration of denaturant; double-stranded fragments made up of mismatched alleles have segments that melt more rapidly, causing such fragments to migrate differently as compared to perfectly complementary sequences (Sheffield et al., “Identifying DNA Polymorphisms by Denaturing Gradient Gel Electrophoresis” in Innis et al., supra, 1990).

Other molecular methods useful for determining the presence or absence of a SNP and/or a haplotype are known in the art and useful in the methods of the invention. Other well-known approaches for determining the presence or absence of a SNP and/or a haplotype include automated sequencing and RNAase mismatch techniques (Winter et al., Proc. Nati, Acad. Sci. 82:7575-7579 (1985)). Furthermore, one skilled in the art understands that, where the presence or absence of multiple alleles or haplotype(s) is to be determined, individual alleles can be detected by any combination of molecular methods. See, in general, Birren et al. (Eds.) Genome Analysis: A Laboratory Manual Volume 1 (Analyzing DNA) New York, Cold Spring Harbor Laboratory Press (1997). In addition, one skilled in the art understands that multiple alleles can be detected in individual reactions or in a single reaction (a “multiplex” assay). In view of the above, one skilled in the art realizes that the methods of the present invention for diagnosing or predicting susceptibility to or protection against CD in an individual may be practiced using one or any combination of the well known assays described above or another art-recognized genetic assay.

One skilled in the art will recognize many methods and materials similar or equivalent to those described herein, which could be used in the practice of the present invention. Indeed, the present invention is in no way limited to the methods and materials described. For purposes of the present invention, the following terms are defined below. As apparent to one of skill in the art, any number of biomarkers may be used in conjunction with various embodiments described herein. Some examples of biomarkers include, but are not limited to, polypeptides, antigens such as glycosylated subunits and lipids, and polynucleotides including microRNA, microsatellite DNA, SNPs, and both genetic and epigenetic. Similarly, it will also be readily apparent to one of skill in the art that the invention can be used in conjunction with a variety of phenotypes, such as serological markers, additional genetic variants, biochemical markers, abnormally expressed biological pathways, and variable clinical manifestations. Finally, one of skill in the art would recognize that the invention can be applied to various metabolic traits, conditions and diseases besides that of metabolic syndrome, insulin resistance and/or elevated liver enzyme levels.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Genetic Variants in the Lipoprotein Lipase Gene

Elevated liver enzyme (LE) levels have been associated with the insulin resistance syndrome (IRS), but the common genetic basis underlying IRS and LE has not been established. Heritability analyses indicate significant evidence for a genetic contribution to LE levels, and co-heritability analyses showed that LE levels share common genetic determinants with IRS in several studies. The lipoprotein lipase (LPL) gene has been shown to be associated with IR in two different cohorts of Hispanic Americans (HA). The fact that the LPL gene was associated with both Gamma-glutamyl transferase (GGT) and IR in HA families recruited through the Insulin Resistance Atherosclerosis Study (IRAS) Family Study has suggested LPL as a common gene underlying GGT levels and IR. The inventors here the role of genetic variants in the LPL gene on GGT levels using 618 non-diabetic offspring from 160 HA families ascertained through a proband with hypertension. GGT was measured by enzymatic colorimetry. Six single nucleotide polymorphisms (SNPs) known to be in the same block in the LPL gene were genotyped in these samples. The generalized transmission disequilibrium test as implemented in the QTDT program was used in the association analysis. To avoid false positives derived from population stratification, the within family variance component was used for the association testing. After adjusting for age, sex, and body mass index, significant association with GGT was found for SNP Ser447Stop/rs328 (p=0.019). An example of rs328 is described herein as SEQ. ID. NO.: 1. Haplotype analysis revealed that the SNP was located at the fourth most common haplotype (GAGGGG), which was also significantly associated with decreased GGT (28.5±2.6 vs 32.2±1.2 U/L, p=−0.009). This haplotype has been previously reported as significantly associated with IRS in HA families recruited through CAD probands (Goodarzi et al., Diabetes, 53:214-220, 2004). These results confirmed that the LPL gene is a common genetic determinant for LEs and IRS in the Hispanic American population.

### Example 2

Genetic Variants in the Lipoprotein Lipase Gene

### LPL Single Marker and Haplotype Frequencies (Table 1)

TABLE 1
SNPs and haplotypes at the LPL locus

| | SNPs and major allele frequencies | | | | | | | |
|---|---|---|---|---|---|---|---|---|
| | 7315 | 8292 | 8393 | 8852 | 9040 | 9712 | | |
| | G→C | A→C | T→G | T→G | C→G | G→A | | Frequency |
| | 0.89 | 0.85 | 0.80 | 0.78 | 0.93 | 0.88 | Chromosomes | (%) |
| Haplotype 1 | G | A | T | T | C | G | 206 | 62.8 |
| Haplotype 2 | G | C | T | T | C | G | 50 | 15.2 |
| Haplotype 3 | C | A | G | G | C | A | 33 | 10.1 |
| Haplotype 4 | G | A | G | G | G | G | 22 | 6.7 |
| Haplotype 5 | G | A | G | G | C | A | 8 | 2.4 |
| Haplotype 6 | G | A | T | G | C | G | 6 | 1.8 |
| Haplotype 7 | C | A | G | G | C | G | 2 | 0.5 |
| Haplotype 8 | G | A | G | G | C | G | 1 | 0.3 |

### Example 3

### Lipin-1 Genetic Variation and Liver Function and Inflammation

Lipin-1 influences adipogenesis and insulin sensitivity in adipose tissue and the liver. It was initially identified as the locus responsible for the fatty liver dystrophy (fld) mouse, which is characterized by absence of adipose tissue depots throughout the body, transient neonatal fatty liver, and peripheral neuropathy. As a key factor in adipogenesis, human adipose lipin-1 mRNA levels are inversely correlated with whole-body insulin resistance, suggesting that by moving fat into adipose tissue, lipin-1 maintains insulin sensitivity by preventing fatty infiltration of liver and skeletal muscle. Furthermore, lipin-1 mRNA levels have been found to be inversely correlated with adipose tissue expression of inflammatory cytokines. Thus, the inventors performed a study to determine whether variants in the gene for lipin-1 (LPIN1) were associated with the liver enzyme gamma glutamyl transferase (GGT, a marker for fatty liver) or inflammatory markers (C-reactive protein, serum tumor necrosis factor (TNF) receptor 1 (TNFR1) and receptor 2 (TNFR2)). The study cohort consisted of 618 non-diabetic offspring from 160 Hispanic-American families ascertained through a proband with hypertension. Two SNPs on opposite ends of the LPIN1 gene were genotyped, haplotypes constructed, and tested for association using generalized estimating equations (GEE1) to account for familial correlation, adjusting for age, sex, and BMI. Haplotype 1 (most common haplotype) was associated with an increase in GGT (haplotype carriers 31.7±1.1 vs non-carriers 29.8±5.1 LPL, p=0.026). SNP rs11524 was associated with decreased TNFR1 (1.77±0.035 vs 1.83±0.020 ng/mL, dominant model, p=0.029). Haplotype 2, which carries rs11524, exhibited the same association. Computational modeling suggests that rs11524 alters an exonic splicing silencer sequence (Ong K L, et al. Am J Hypertens 2008; 21:539-45). Consistent with predictions based on the biology of lipin-1, variants in the LPIN1 gene modulate liver function and inflammation.

### Example 4

### Lipin-1 Genetic Variation and Liver Function and Inflammation

### LPIN1 Haplotypes and SNPs (Table 2)

TABLE 2
LPIN1 haplotypes and SNPs

| | rs893347 (SEQ. ID. | rs11524 (SEQ. ID. |
|---|---|---|
| | NO.: 2) | NO.: 3) |
| Haplotype 1 | C allele | T allele |
| Haplotype 2 | C allele | C allele |
| Haplotype 3 | G allele | T allele |

While the description above refers to particular embodiments of the present invention, it should be readily apparent to people of ordinary skill in the art that a number of modifications may be made without departing from the spirit thereof. The presently disclosed embodiments are, therefore, to be considered in all respects as illustrative and not restrictive.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventor that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. Furthermore, it is to be understood that the invention is solely defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Accordingly, the invention is not limited except as by the appended claims.

1. A method of diagnosing susceptibility to insulin resistance in an individual, comprising:
determining the presence or absence in the individual of a risk variant at the Lipoprotein Lipase (LPL) genetic locus and/or Lipin-1 (LPIN1) genetic locus;
determining the presence or absence in the individual of an elevated level of a marker for fatty liver; and
diagnosing susceptibility to insulin resistance in the individual based upon the presence of the risk variant at the LPL genetic locus and/or LPIN1 genetic locus and the presence of the elevated level of the marker for fatty liver.

2. The method of claim 1, wherein the risk variant at the LPL genetic locus comprises SEQ. ID. NO.: 1.

3. The method of claim 1, wherein the risk variant at the LPIN1 genetic locus comprises SEQ. ID. NO.: 2 and/or SEQ. ID. NO.: 3.

4. The method of claim 1, wherein the individual is Hispanic American.

5. The method of claim 1, wherein the marker for fatty liver comprises GOT.

6. A method of determining a low probability of developing insulin resistance in an individual, comprising:
determining the presence or absence in the individual of a protective haplotype at the Lipin-1 (LPIN1) genetic locus;
determining the presence or absence of a low level of expression of an inflammatory marker; and
diagnosing a low probability of developing insulin resistance in the individual based upon the presence of a protective haplotype at the LPIN1 genetic locus and the presence of a low level of expression of the inflammatory marker.

7. The method of claim 6, wherein the inflammatory marker comprises tumor necrosis factor receptor 1 (TNFR1).

8. The method of claim 6, wherein the protective haplotype at the LPIN1 genetic locus comprises SEQ. ID. NO.: 2 and/or SEQ. ID. NO.: 3.

9. The method of claim 6, wherein the individual is Hispanic American.
